(19) United States
(12) Patent Application Publication
(10) Pub. No.: US 2007/0128320 A1
(43) Pub. Date: Jun. 7, 2007

(54) QUANTITATIVE TEST METHOD FOR DETERMINING HEAT TRANSFER OIL IN EDIBLE OIL OR FAT

(76) Inventor: Masami KUBOTA, Tokyo (JP)

Correspondence Address: SHELL OIL COMPANY, P O BOX 2463, HOUSTON, TX, 772522463, US

(21) Appl. No.: 11/558,290

(22) Filed: Nov. 9, 2006

(30) Foreign Application Priority Data
Nov. 11, 2005 (JP) 2005-327246

(51) Int. Cl. G01N 33/02

(52) U.S. Cl. 426/231

(57) ABSTRACT

A quantitative test method for determining the content of a heat transfer oil mixed with a polar edible oil or fat, wherein the polar edible oil or fat containing a trace amount of the heat transfer oil is diluted with a nonpolar organic solvent, said edible fat or oil is adsorbed onto a column packing medium by passing the dilute solution thereof through a separation column, the heat transfer oil is allowed to flow out from the separation column together with the nonpolar organic solvent, the solution that has thus flowed out is concentrated and subjected to gas chromatography, and a quantitative determination is performed by comparison with the gas chromatography analysis value of a reference solution of heat transfer oil.

### RELATED CASES

The present application claims priority from Japanese Application No. 2005-327246, filed Nov. 11, 2005.

### FIELD OF THE INVENTION

The present invention relates to a quantitative test method for determining the content of a heat transfer oil mixed with an edible oil or fat, and a method of monitoring the transfer oil using this quantitative method.

### BACKGROUND OF THE INVENTION

In factories that produce edible oils or fats, the production process requires heat treatment to be performed. Ordinarily, this heat treatment is performed by using a heat transfer oil. It is necessary, and law in some countries, from the point of view of food product hygiene, to prevent admixture or even traces of heat transfer oil with such edible oils or fats. Such admixtures may occur, for example, due to causes such as pinhole leaks resulting from ageing of the equipment.

Also, in a food plant or the like which performs frying processing of various types of foods, cakes or the like with such edible oils or fats, in the process of heating the edible oil or fat, heating is performed by a heating apparatus using heat transfer oil. Although the edible oils or fats and the heat transfer oil are of course separated from each other inside the heating apparatus, the possibility cannot be avoided that some of the heat transfer oil may be mixed via pin hole leaks with the edible oils or fats.

Accordingly, in view of food hygiene, in a factory that produces edible oil or fat, or a food plant or the like, a safe heat transfer oil that is free of toxicity is used. However, even when using a heat transfer oil having a high level of safety, it is absolutely essential to make the degree of mixing of the heat transfer oil into the edible oil or fat as small as possible. Hence, the quantity of the heat transfer oil that gets mixed with the edible oils or fats is frequently determined, in fact for every production lot, so as to make it possible to respond to mixing of even a trace amount of the heat transfer oil with the edible oils or fats.

In a conventional quantitative test method an edible oil or fat sample is saponified by potassium hydroxide, diluted in alcohol as a solvent and then thoroughly shaken in a mixed solution of water and hexane to extract the heat transfer oil into the hexane layer and the saponified edible oil or fat into the water layer. The heat transfer oil recovered in the hexane layer is then subjected to a quantitative determination by gas chromatography.

At present, in an edible oil or fat producing factory or food plant, an independent control reference is set up such that the amount of the heat transfer oil to be mixed into the edible oil or fat is controlled to be less than 100 ppm. This control reference value is based on the lower limiting value of a conventional quantitative test method as described above. However, given that the allowed concentration of food grade heat transfer oils in food is <10 ppm it will be appreciated that this level of measurement is not acceptable.

Further, it is known in Japanese Laid-open Patent Application No. 2004-108844 to perform evaluation of edible oil or fat using gas chromatography for analysis of a trace amount of a volatile component in the edible oil or fat and then to perform a sensory evaluation by comparing the analytical value obtained by such gas chromatography with a sensory examination value obtained by a group of panelists.

It is highly desirable to be able to provide edible oils, fats and food products of greater safety by facilitating control of oil or fat in food products by more convenient and rapid analysis and accurate quantitative determination, even at low concentrations of heat transfer oil mixed with edible oil or fat.

### SUMMARY OF THE INVENTION

According to the present invention, there is surprisingly provided an advantageous quantitative test method for determining the content of a heat transfer oil mixed with a polar edible oil or fat, wherein the polar edible oil or fat containing a trace amount of the heat transfer oil is diluted with a nonpolar organic solvent, said edible oil or fat is adsorbed onto a column packing medium by passing the dilute solution thereof through a separation column, the heat transfer oil is allowed to flow out from the separation column together with the nonpolar organic solvent, the solution that has thus flowed out is concentrated by removing organic solvent and subjected to gas chromatography, and a quantitative determination is performed by comparison with the gas chromatography analysis value of a reference solution of heat transfer oil.

Said analysis maybe performed by finding the peak area value of the heat transfer oil in the gas chromatograph, and comparing this with the peak area value of a reference solution containing heat transfer oil analysed in the same way.

### BRIEF DESCRIPTION OF THE DRAWINGS

For a more detailed understanding of certain embodiments, reference will be made to the accompanying Figures, wherein:

FIG. 1 is a plot of a gas chromatographic analysis for the samples of Example 1; and

FIG. 2 is a plot of a gas chromatographic analysis for the samples of Example 2.

### DETAILED DESCRIPTION OF THE PREFERRED EMBODIMENTS

In the embodiment, by the use of a separation column, a certain polar edible oil or fat and a heat transfer oil can be efficiently separated. By subjecting the heat transfer oil that has thus been separated to gas chromatography and comparing with the peak area of reference solutions, analysis can be achieved in a rapid and straightforward fashion, making it possible to achieve quantitative determination of the content of a heat transfer oil mixed with a polar edible oil or fat with excellent accuracy. Also, by this quantitative test method, the condition of the edible oil or fat can easily be monitored.

In other aspects, the present invention further provides a method of monitoring a heat transfer oil wherein: reference solutions of polar edible oil or fat containing respective contents of a plurality of heat transfer oils are diluted with a nonpolar organic solvent; said edible oil or fat is adsorbed on a column packing medium by allowing the dilute solution thereof to pass through a separation column; said heat transfer oil is allowed to flow out of the separation column together with the nonpolar organic solvent; the solution that has thus flowed out is concentrated; the peak area of each heat transfer oil is determined by subjecting the thus-concentrated solution to gas chromatography; the peak area of gas chromatography obtained by processing edible oil or fat having an unknown content of the heat transfer oil in the same way as said reference solutions is compared with the peak area of said reference solutions; and the state of the edible oil or fat is thus monitored by determining the unknown content of heat transfer oil therein.

Although the edible oil or fat, being constituted by glycerides of fatty acids, is polar, preferably the organic solvent that is employed for this edible oil or fat is a nonpolar organic solvent: for example pentane, hexane or heptane or the like. In particular, nonpolar hydrocarbons having a carbon number in the range of from 5 to 7 may be conveniently employed. The organic solvent is preferably employed in a volume amount of in the range of from 50 to 200 times the volume amount of polar edible oil or fat.

The polar edible oil or fat containing a trace amount of heat transfer oil dissolved in the organic solvent is separated into edible oil or fat and heat transfer oil using the separation column.

The column packing medium that is packed in the separation column is preferably a substance capable of adsorbing polar edible oil or fat. For example, said column packing medium may be conveniently selected from one or more of aluminium and silicate minerals, Attapulgus clay, alumina or silica gel.

In one embodiment of the present invention, a multi-layered packing column may be employed in which the above packing agents are used in combination, such as for example, a multi-layer packing column of Attapulgus clay and alumina. An even better separating effect can thereby be obtained, making it possible to further raise the accuracy of the quantitative test method.

The separation column is preferably formed in an appropriate size: for example, the internal diameter of the separation column may be conveniently in the range of from 20 to 30 mm and the length of said separation column may be conveniently in the range of from 70 to 130 cm.

After the above separation column has been prepared by washing (for example, with dichloromethane and/or the nonpolar organic solvent), the polar edible oil or fat may be dissolved in the above organic solvent and may be added gradually to the top of the column.

As the solution of polar edible oil or fat and organic solvent descends through the separating column, the polar edible oil or fat is adsorbed onto the column packing medium, while the heat transfer oil flows out from the separation column together with the nonpolar organic solvent in a condition wherein the heat transfer oil is dissolved in the nonpolar organic solvent. The heat transfer oil can thus be recovered from the polar edible oil or fat.

The solution of heat transfer oil and nonpolar organic solvent that flows out of the separation column as mentioned above is concentrated in order to remove the organic solvent.

The solution that has flowed out of the separation column may be concentrated to a degree such as to make it easy to perform analysis using gas chromatography: for example, the solution may be conveniently concentrated to an amount in the range of from 0.1 to 3 ml.

This concentrated solution may be analysed by being subjected to gas chromatography by the ordinary method and the total peak area value representing the heat transfer oil may be found from an analysis chart.

Concurrently, reference solutions of edible oil or fat containing known quantities of heat transfer oil are subjected to gas chromatography by processing in the same way, and the peak area of the heat transfer oil is found from an analysis chart based on the reference solutions of heat transfer oil. Thus, reference solutions having a plurality of concentrations are used as these reference solutions.

The content of heat transfer oil admixed with the edible oil or fat can be found by comparing the total peak area obtained from the gas chromatography analysis chart including heat transfer oil of unknown content as mentioned above with the total peak area value based on the plurality of reference solutions including fixed contents of heat transfer oil.

In this way, the content of heat transfer oil admixed with the edible oil or fat can be found by comparison with the reference solutions of heat transfer oil, so the edible oil or fat can be maintained in an optimum condition and can be controlled by watching this content as an index.

Various known types of heat transfer oil can be employed as the heat transfer oil in the edible oil or fat that is the subject of quantitative test method referred to above. One of the most preferable types of these heat transfer oils is petroleum-based hydrocarbon oil. Of such heat transfer oils, in particular liquid paraffin is recognised as a food additive and as a mould release agent for cakes and bread, and is therefore preferred on account of its high degree of safety.

### EXAMPLES

The present invention is described below with reference to the following Examples which are not intended to limit the scope of the present invention in any way.

### Example 1

The procedure of Example 1 is described below.

(i) Preparation of Sample

Samples of three types were prepared, namely, (a) a sample in which a typical commercial heat transfer oil constituted of liquid paraffin was added in a concentration of 33 ppm in 1 g of edible oil or fat comprising medium-chain fatty acid triglyceride, (b) a sample in which said heat transfer oil was added in a concentration of 100 ppm in 1 g of said edible oil or fat, and (c) a sample in which no heat transfer oil was added (blank (0 ppm)) to said edible oil or fat.

1 g of each of these samples (a)-(c) was diluted respectively with pentane to 100 ml in each case.

The chief constituents of the above medium-chain fatty acids were fatty acids having a carbon number in the range of from 6 to 12.

(ii) Separation Column

A glass column of length 100 cm fitted with a stopcock made of a fluorine resin and internal diameter 25 mm was prepared; this glass column was used as a separation column by packing it with 120 g of Attapulgus clay (aluminium silicate mineral).

First, 100 ml of dichloromethane was allowed to flow through this separation column. Next, the column was washed by passing 150 ml of pentane therethrough, leaving a minute amount of pentane at the top of the column; the aforementioned stopcock was closed prior to exposure of the column packing medium to air, and the liquid that flowed through it was discarded.

(iii) Separation Operation

The following separation operation, concentration operation and gas chromatographic analysis was followed for each of the aforementioned samples (a)-(c):

A test-tube was placed below the above separation column for collection purposes and the stopcock was opened. The liquid obtained by washing through with pentane was recovered, adding one of the aforementioned sample solutions in a stepwise fashion in lots of about 5 ml in each case. After all of the 100 ml of the solution had been allowed to flow through the separation column, a further lot of about 5 ml in each case of pentane was added, and the separation operation was terminated by closing the stopcock at the time-point where 120 ml of the liquid washed through by the pentane had been recovered.

(iv) Concentration Operation

The aforementioned test-tube containing 120 ml of the pentane solution flowing out of the separation column was inserted in a water bath at 30° C.; the pentane was evaporated off by gently blowing in nitrogen gas, thereby achieving concentration to 1 ml.

(v) Gas Chromatographic Analysis

As the gas chromatographic equipment, an “HP6890” machine manufactured by Hewlett-Packard Inc was employed. The column used was an “Ultra alloy-SIM” (5 m ×0.53 mm ×0.1 μl) manufactured by Frontier Laboratories Inc; the carrier gas was helium, of pressure 1.3 psi, and the method of injection was splitless injection, the quantity injected being 1 μl. An FID detector was employed to conduct gas chromatographic analysis.

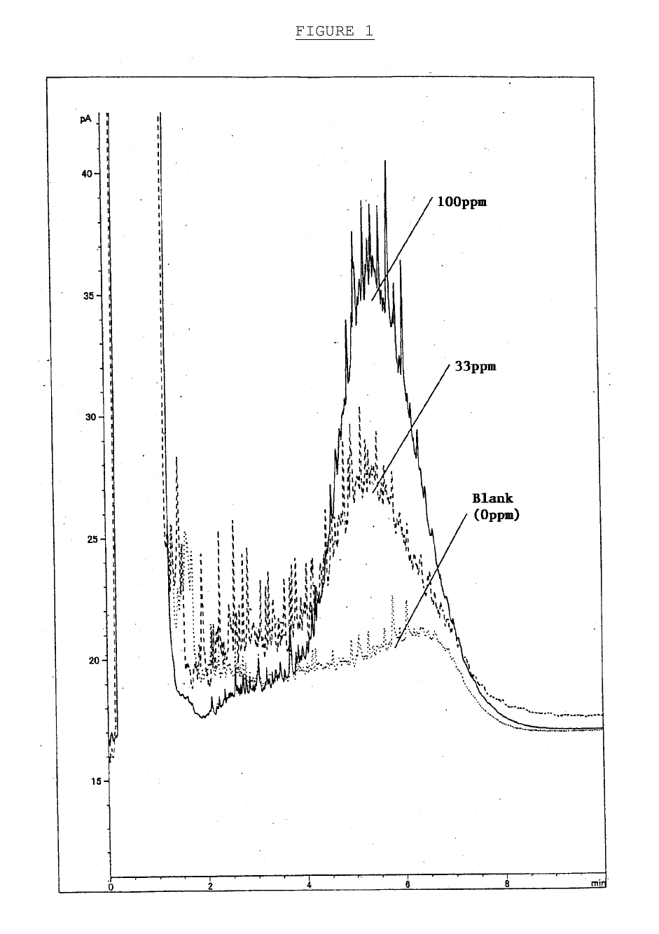

FIG. 1 shows a chart of the gas chromatographic analysis results for each of the three samples (a)-(c) of Example 1.

In the gas chromatographic analysis chart, FIG. 1, the presence of the liquid paraffin heat transfer oil was shown by a peak between 4 min to 7 min.

(vi) Analysis Results

The above gas chromatographic chart was used to find the peak area value of a reference solution containing 33 ppm of liquid paraffin, the peak area value of a reference solution containing 100 ppm of liquid paraffin, and the peak area value of a reference solution not containing liquid paraffin (blank (0 ppm)).

The results are as shown in Table 1 below.

The gas chromatograph analysis was conducted three times. FIG. 1 shows the analysis chart for the first of these runs in Table 1. Table 1 also shows the average values obtained by combining the results of these three analyses.

TABLE 1

| | Added amount | | |
|---|---|---|---|
| No. of times | of liquid | | Average |
| of analysis | paraffin | Area value | value |
| (1) | (Blank(0 ppm)) | 818 | |
| (2) | (Blank(0 ppm)) | 1016 | |
| (3) | (Blank(0 ppm)) | 1204 | (1012) |
| (1) | 33 ppm | 1906 | |
| (2) | 33 ppm | 1863 | |
| (3) | 33 ppm | 1982 | (1917) |
| (1) | 100 ppm | 2443 | |
| (2) | 100 ppm | 2209 | |
| (3) | 100 ppm | 2364 | (2339) |

(vii) Examination of Results

As is clear from Table 1 and FIG. 1, which show the above analysis results, clear differences are seen in the area values in the cases where the contents of liquid paraffin heat transfer oil were (blank (0 ppm)), 33 ppm and 100 ppm, respectively.

It can thus be seen that it is possible to measure the contents of heat transfer oil in edible oil or fat by comparing the total peak area values with the peak area values of the above reference solutions.

### Example 2

(i) Preparation of Sample

Samples of five types were prepared, namely, samples in which heat transfer oil constituted of liquid paraffin was added in a concentration of (blank (0 ppm)), 10 ppm, 20 ppm, 33 ppm and 100 ppm, respectively, in 1 g of edible oil or fat comprising medium-chain fatty acid triglyceride as referred to above. The rest of the experimental procedure was the same as in Example 1.

(ii) Separation Column

A glass column of length 100 cm fitted with a stopcock made of a fluorine resin and internal diameter 25 mm was prepared; this glass column was used as a separation column by packing it with 10 g of alumina gel at the bottom of the column and 100 g of Attapulgus clay (aluminium silicate mineral) on top of the alumina gel, thereby forming two layers.

First of all, 100 ml of dichloromethane was allowed to flow through this separation column in the same way as in Example 1. Next, the column was washed by passing 150 ml of pentane therethrough, leaving a minute amount of pentane at the top of the column; the aforementioned stopcock was closed prior to exposure of the column packing medium to air, and the liquid that flowed through it was discarded.

The separation operation (iii), concentration operation (iv) and gas chromatographic analysis (v), respectively, were conducted in the same way as in Example 1.

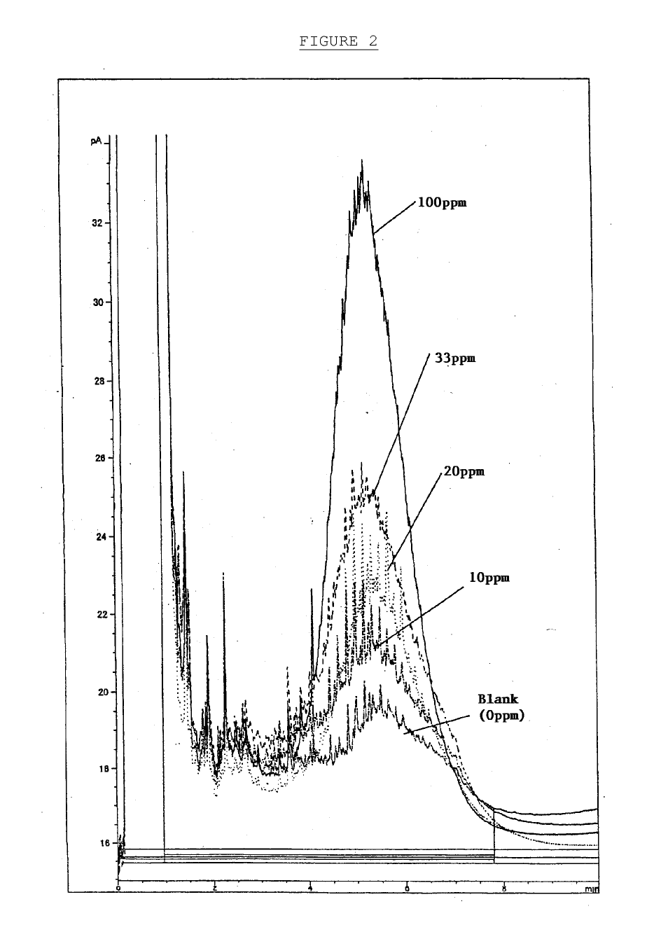

FIG. 2 shows a chart of the results of gas chromatographic analysis for each of the five samples of Example 2.

(vi) Analysis Results

The gas chromatographic analysis chart shown in FIG. 2 was used to find the peak area values of a reference solution containing 0 ppm (blank) of liquid paraffin, a reference solution containing 10 ppm of liquid paraffin, a reference solution containing 20 ppm of liquid paraffin, a reference solution containing 33 ppm of liquid paraffin, and a reference solution containing 100 ppm of liquid paraffin.

The results are as shown in Table 2 below.

TABLE 2

| Added amount of liquid | |
|---|---|
| paraffin | Area value |
| (Blank(0 ppm)) | 268 |
| 10 ppm | 535 |
| 20 ppm | 620 |
| 33 ppm | 1057 |
| 100 ppm | 1639 |

(vii) Examination of Results

As is clear from FIG. 2 and Table 2, which show the above analysis results, clear differences are seen in the area values in the cases where the contents of liquid paraffin heat transfer oil were (blank (0 ppm)), 10 ppm, 20 ppm, 33 ppm and 100 ppm, respectively.

It can thus be seen that it is possible to perform measurement even of contents as low as 10 ppm or 20 ppm of heat transfer oil by employing an combination of Attapulgus clay and alumina gel in a separating column as described above.

1. A quantitative test method for determining the content of a heat transfer oil mixed with a polar edible oil or fat, comprising:
a) diluting the polar edible oil or fat containing the heat transfer oil with a nonpolar organic solvent to form a dilute solution;
b) absorbing the edible fat or oil onto a column packing medium by passing the dilute solution through a separation column;
c) allowing a solution containing heat transfer oil and nonpolar organic solvent to flow out of the separation column;
d) concentrating the solution that flows out of the separation column to produce a concentrated solution;
e) subjecting the concentrated solution to gas chromatography to produce a gas chromatography analysis; and
f) performing a quantitative determination by comparing the gas chromatography analysis with a gas chromatography analysis value of a reference solution of heat transfer oil.

2. The quantitative test method according to claim 1, wherein the heat transfer oil is liquid paraffin.

3. The quantitative test method according to claim 1, wherein the nonpolar organic solvent is a hydrocarbon having a carbon number in the range of from 5 to 7.

4. The quantitative test method according to claim 1, wherein the column packing medium of said separation column is selected from one or more of aluminium and silicate minerals, Attapulgus clay, alumina and silica gel.

5. The quantitative test method according to claim 1, wherein the column packing medium of said separation column is a multilayer packing of Attapulgus clay and alumina.

6. A method of monitoring a heat transfer oil wherein: reference solutions of polar edible oil or fat containing respective contents of a plurality of heat transfer oils are diluted with a nonpolar organic solvent; said edible oil or fat is adsorbed on a column packing medium by allowing the dilute solution thereof to pass through a separation column; said heat transfer oil is allowed to flow out of the separation column together with the nonpolar organic solvent; the solution that has thus flowed out is concentrated; the peak area of each heat transfer oil is determined by subjecting the thus-concentrated solution to gas chromatography; the peak area of gas chromatography obtained by processing edible oil or fat having an unknown content of the heat transfer oil in the same way as said reference solutions is compared with the peak area of said reference solutions; and the state of the edible oil or fat is thus monitored by determining the unknown content of heat transfer oil therein.
